(19) United States
(12) Patent Application Publication
(10) Pub. No.: US 2015/0201900 A1
(43) Pub. Date: Jul. 23, 2015

(54) MULTI-PANE IMAGING TRANSDUCER ASSOCIATED WITH A GUIDEWIRE

(71) Applicant: Mubin I. Syed, Spingfield, OH (US)

(72) Inventor: Mubin I. Syed, Spingfield, OH (US)

(21) Appl. No.: 14/676,774

(22) Filed: Apr. 1, 2015

Related U.S. Application Data
(63) Continuation-in-part of application No. 13/750,920, filed on Jan. 25, 2013.
(60) Provisional application No. 61/590,472, filed on Jan. 25, 2012.

(51) Int. Cl. A61B 8/08; A61M 25/09; A61M 25/01; A61B 8/00; A61M 25/06; A61F 2/95; A61B 8/14

(52) U.S. Cl. CPC A61B 8/0841; A61F 2/95; A61M 25/09041; A61B 8/145; A61B 8/4494; A61B 8/0891; A61M 25/065; A61B 8/5207; A61B 8/463; A61M 25/0108; A61M 2025/09116

(57) ABSTRACT

A multi-plane imaging transducer generates a plurality of planes of image data from the ultrasound echo information including at least a first plane, a second plane orthogonal to the first plane, and a third plane orthogonal to the first and second planes. The multiplane imaging transducer may further generate a transverse view, sagittal view, and coronal view from derived from previously stated image planes. The transducer is used in association with a guidewire inside a superficial artery, such as the superficial temporal artery. The guidewire has a distal end formed into a knob and has a bend for steering through the artery. The knob is used to allow snaring for mechanical engagement from a femoral artery catheter.

### CROSS REFERENCE TO RELATED APPLICATIONS

The present patent application is a continuation-in-part application of co-pending U.S. patent application Ser. No. 13/750,920 entitled “Method and Apparatus for Percutaneuous Superficial Temporal Artery Access for Cartoid Artery Stenting”, filed Jan. 25, 2013, and which claims priority to U.S. Provisional Patent Application No. 61/590,472, filed Jan. 25, 2012, the entireties of which are herein incorporated by reference.

### TECHNICAL FIELD

The present invention relates to an imaging device which enables the placement of a guidewire which is prerequisite to the installation of a stent in the carotid artery of a patient with a hostile aortic arch. In particular, the invention relates to the use of multi-plane ultrasound imaging to allow the introduction of a guidewire into the Superficial Temporal Artery (STA), the STA guidewire being subsequently snared by a guidewire introduced into the femoral artery and guided to the aortic_arch or carotid artery.

### BACKGROUND

FIG. 1 shows a subset of the arterial vascular system of a subject 102 in need of a stent, and for clarity in understanding the issues related to guiding the stent into a human patient, the venous system, organs, pulmonary arteries and veins, and many branches of the circulatory system are not shown. Heart 104 pumps oxygenated blood through the aortic arch 106, which guides upward-flowing blood from the heart to a downward flow for delivery to the lower organs and right femoral artery 120 and left femoral artery 122, which are interchangeably used for stent arterial access, as will be described later. Many variations in the vessels formed in a subject are found from one subject to another, and the particular subject 102 shown in FIG. 1 has an aortic arch 106 with three major branching vessels which leave the aortic arch, including a first branching vessel which forms the left subclavian artery (LSA) 114 and left vertebral artery (LVA) 116, a second branching vessel which forms the left common carotid artery 118 (LCCA), and a third branching vessel which forms the right subclavian artery (RSA) 108, right vertebral artery (RVA) 110, and right common carotid artery (RCCA) 112. Arteriosclerotic disease processes known as Atherosclerosis often afflict the arterial system, and the affected areas include the aortic arch 106, left common carotid artery 118 (branching to internal carotid artery 119 and external carotid artery 121), and right common carotid artery 112 (which also branch to internal and external carotid arteries, not shown) The disease processes which take place in these vessels cause deterioration of the interior vessel walls, and diseased material which detaches from the interior vessels can be swept through the arterial system with successively decreasing vessel diameter until it becomes lodged in a vessel constriction, causing the cessation of blood flow in the blocked area, leading to tissue death from loss of oxygenation. This disease process is the leading cause of strokes, heart attacks, and other debilitating or fatal events. When a subject presents with this disease process, a variety of imaging techniques may be used to ascertain the nature of the blockage or potential blockage, using contrast agents and x-ray (conventional C-arm angiography), magnetic resonance (MR) imaging (MR angiography), or computerized axial tomography (CAT) scans (CT angiography), and whereby the imaging contrast agent provides increased differentiation between the vessel walls and the blood flowing through the vessel.

As atherosclerosis in the carotid artery progresses, the risk of stroke increases, and it becomes necessary to intervene to prevent stroke or death from clots or vessel debris which becomes lodged in the brain, specifically related to disease of the internal carotid artery branch which serves the brain, or the common carotid artery which precedes it in the circulatory path. It should be noted that stroke is the third leading cause of death in the developed nations. 85% of all strokes are ischemic (due to brain circulation compromise) in nature and 20-30% of all ischemic strokes are caused by carotid artery atherosclerotic occlusive disease. For atherosclerotic occlusive disease of the internal or common carotid artery, one procedure performed by interventionalists (interventional radiologists, vascular surgeons, or interventional cardiologists) is the installation of a stent, which is an expanding cylindrical wire or plastic mesh which supports and stabilizes the diseased area of the artery, and reduces the stenosis (narrowing) of the artery through a treatment known as angioplasty, whereby an inflatable balloon is used to momentarily expand the stent across the inner diameter of the vessel in the stenotic region.

The prior art installation of a carotid artery stent described in relation to FIG. 1 is done in a series of steps for which the order of the steps and types of equipment may vary. As will be described, the types of interventional devices which are used in the procedures include a small diameter guidewire which may be co-inserted with a small catheter, a subsequently inserted stiff guidewire which may be co-inserted or replace the small diameter guidewire in the small catheter, a sleeve or sheath which may be threaded over the small catheter, and an angioplasty catheter which may be threaded over the small catheter or exchanged for the small catheter during the procedure. For clarity, FIG. 1 broadly indicates the path 135 used by interventional devices in the example (where the interventional devices may represent any of the previously described devices in any combination and achieving an insertion level which is typically less than the entire path length 135), which extends to the ultimate treatment region 136 reached by the stent catheter.

Following FIG. 1, the first step of a stenting procedure involves threading the small diameter guidewire, which may be a 0.035 inch diameter steerable guidewire having a bent tip (where the tip may also be hydrophilic), through a steerable catheter which may also have a bent tip for steering. Examples of such steerable catheter guidewires are Berenstein or Vertebral with trade names HIH or Headhunter) or reverse curve (Simmons or Vitek). Steering is done by rotating the guidewire, which causes the bent tip to select the desired artery and follow that arterial path with the catheter following and assisting in selection. The second step therefore is the installation at percutaneous location 132 of a femoral artery (FA) catheter, which is usually 4 to 6 French diameter, and guided into the femoral artery through the aortic arch 106 and to the common carotid artery 118. The guidewire is fed from a sterile drape 130 which furnishes the approximately 260 cm of length required. The external carotid artery (ECA) 121 is then selected often with the angled FA catheter and same guidewire followed by selection of an external carotid artery branch while the guidewire is advanced.

Navigational information on the progress of the guidewire is provided by a radiographic display which is used in combination with arterial contrast agents which delineate the vessel walls with respect to the guidewire. One typical imaging system is x-ray fluoroscopy, whereby a source of x-rays is applied in one or more planes through the patient to a 2D or 3D detector, and the real-time radiographic images are used by the interventionalist to provide guidance information. The small diameter guidewire inside the catheter is then replaced with a stiff guidewire (to eventually support the subsequently placed long guiding sleeve or sheath) The small diameter catheter may or may not be removed at this point leaving the stiff guidewire in place. The long guiding sleeve or sheath (6 to 8 French) is then advanced over the stiff guidewire alone (or over the catheter and stiff guidewire combination) from the femoral access through the descending aorta region 134 and through the aortic arch region 106 to the distal common carotid artery 118 just below the bifurcation. Contrast injection is performed through the guiding sleeve or sheath to now visualize the internal carotid artery 119 and external carotid artery 121. The stiff guidewire in the ECA 121 is typically removed at this point. The stenosis in the internal carotid artery (ICA) 119 is gently traversed with a 0.014 inch guidewire tip fixed embolic protection device (EPD), examples of which are manufactured under the trade names Accunet or Filterwire, versus a embolic protection device that is separately deployed over a 0.014 inch guidewire with a 0.017 inch tip, such as those with trade names Nav6 or Emboshield. The EPD is deployed within the distal portion of the cervical segment of the ICA 119. An angioplasty balloon catheter is then threaded through the sheath over the guidewire portion of the EPD to the location of the stenosis 136 to then predilate the stenosis. The angioplasty catheter is then exchanged for a stent delivery catheter which has a self-expanding stent at the distal end, which is guided to the site of the stenosis 136 shown in FIG. 1. A slightly larger angioplasty balloon catheter is then used to postdilate the stent to the desired diameter. Carotid and cerebral angiography is then perform to confirm an adequate result.

The critical part of steering occurs when selecting the particular vessel of the aortic arch shown in FIG. 1. Each subject 102 undergoing the procedure may have a different aortic arch vascular configuration. FIG. 1 shows a common arrangement of vessels at the aortic arch, as was previously described, and the radiographic contrast agent is injected to delineate the vessel outlines, which enables the interventionalist to select and steer into the vessel which leads to the desired carotid artery, shown as the left internal carotid artery 119 with the expanding stent on the distal end of the stent delivery catheter, which was introduced after the angioplasty catheter as previously described. The stent may be a wire or plastic mesh which is guided into place and affixed by inflating a balloon which deforms the stent to conform to the inner diameter of the blood vessel. Many variations in the arrangement of arteries which branch from the aortic arch may be present, and are classified according to “type” where the type number signifies the extent of vertical deviation (or slope) of the bifurcation point for arteries which branch at the top of the aortic arch. FIG. 1 shows three arteries which branch from the top of the aortic arch 106 in substantially the same horizontal plane, and is known as a “type I” aortic arch. A “type II” aortic arch has one of the branching arteries located 1-2 common carotid artery diameters below the topmost, and a “type III” aortic arch has a separation from horizontal of more than 2 common carotid artery diameters. Since the stent delivery catheter is advanced from the descending aorta region 134 to the top of the aortic arch 106, the type number provides an indication of how difficult the navigation to the common carotid artery will be according to the extent to which the catheter must change direction to guide into the common carotid artery, as will be described. As can be seen from FIG. 1, the long guiding sleeve or sheath can be guided into the left common carotid artery 118 without significant changes in direction along the path. The long guiding sleeve or sheath is typically placed in the common carotid artery 118 just below the bifurcation point for the external carotid artery 121 and internal carotid artery 119, after which the angioplasty catheter and stent delivery catheter are guided into the desired artery such as the internal carotid artery 119 as shown in FIG. 1.

FIG. 2A shows a magnified view of an aortic arch region 202 variation from 106 of FIG. 1, where the subject of FIG. 2A has a type II aortic arch variation (branching artery 211 is 1-2 carotid artery diameters below the first branch 204 indicated by distance 209) in vascular configuration and also a branch point for the LCCA 206 above second branch 211, known collectively as a type II-A Bovine aortic arch 208, which is distinguishable from FIG. 1 having three branches at the top of the aortic arch. The type II-A Bovine aortic arch has only two branches 204 and 211 at the top of the aortic arch, with the left common carotid artery 206 not having its own vessel leading to the aortic arch (as was the case in FIG. 1), but instead branching off from second branch 211 which also forms the right common carotid artery 214, right vertebral artery 212, and right subclavian artery 210. One difficulty that can be seen from the LCCA 206 geometry of FIG. 2A is that the initial guidewire entry into the LCCA from the aortic arch 202 is a sharp turn in a vessel transitioning from the large diameter aortic arch 202 to the much smaller diameter LCCA 206, and additionally with a sharp angle of approach and difficult catheter guidance. This type of Bovine aortic arch can be difficult to navigate without insult or injury to the adjacent inner walls of the aorta, which are likely to also have atherosclerosis as the region to be treated by angioplasty, and this agitation during guidance may cause disease vessel material to break loose and cause a stroke during the installation of the stent—the original purpose of the stenting procedure was to reduce such risk. Aside from during guidance, agitation and stroke may also occur when the sheathe, stent and EPD all migrate retrograde instantaneously or “flip” out of the internal carotid (dragging the EPD through the critical stenosis) and common carotid arteries into the ascending aorta (during actual stent deployment as a result of the relative increased stiffness of the stent and stent delivery system). Blood vessels which present these risks associated with atherosclerosis and with difficult entry geometry are known as hostile vessels, and accordingly, the aortic arch of FIG. 2A is known as a hostile aortic arch, with the aortic arch type classification indicating the level of difficulty and type of difficulty for FA catheter guidance to the carotid artery.

### SUMMARY

An apparatus and method for installation of a carotid artery stent which eases the navigation of the guidewire through hostile vessels of the aortic arch, thereby reducing patient risk and procedure length, and accordingly increasing patient safety, is disclosed herein.

An apparatus and method for through-and-through access and guidance through tortuous vessels by using a major vessel for entry of a catheter into a large vessel in combination with the entry of a guidewire into a minor surface vessel, the apparatus and method for use with or without a multi-plane imaging device for navigating the tortuous vessel region, is also disclosed herein.

Embodiments of the apparatus may be used for endovascular stroke intervention and other neuro-interventional procedures in hostile aortic arches. Also this device can be helpful for quick and reliable radial artery access for any type of Endovascular interventional and pedal artery access for limb salvage procedures.

### BRIEF DESCRIPTION OF THE DRAWINGS

The subject matter that is regarded as the invention is particularly pointed out and distinctly claimed in the claims at the conclusion of the specification. The foregoing and other objects, features and advantages of the invention will be apparent from the following detailed description taken in conjunction with the accompanying drawings.

FIG. 1 is a front view of a human subject with a prior art femoral artery guidewire.

FIG. 2A is a detail view of the aortic arch region of FIG. 1 but with a type IIB variant of the aortic arch.

FIG. 2B is a view of FIG. 2A where an STA guidewire and FA catheter are engaged in accordance with one embodiment of the invention.

FIG. 2C is a detail view of FIG. 2B while snaring in accordance with one embodiment of the invention.

FIG. 2D is a detail view of the aortic arch region of FIG. 1 but with a type III variant of the aortic arch in accordance with one embodiment of the invention.

FIG. 3 is a saggital view of the STA access and ultrasound imaging transducer in accordance with one embodiment of the invention.

FIG. 4 is a perspective view of the imaging planes of the ultrasound transducer of

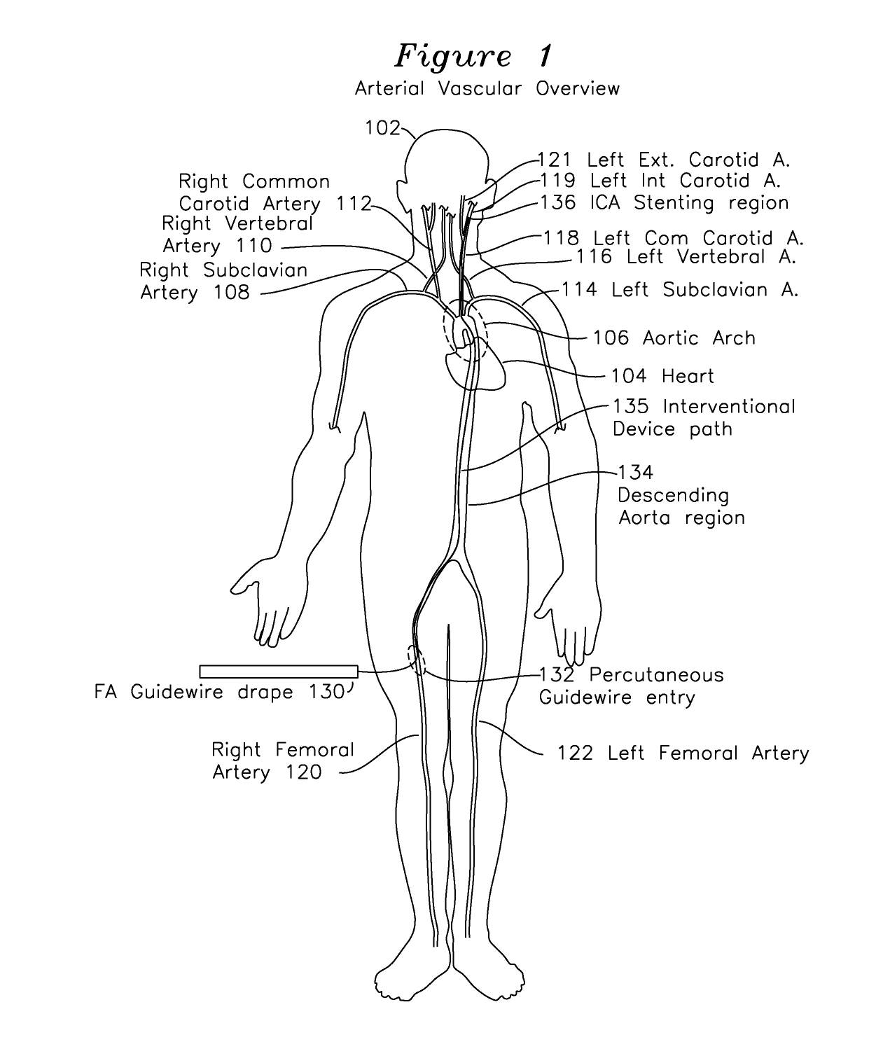

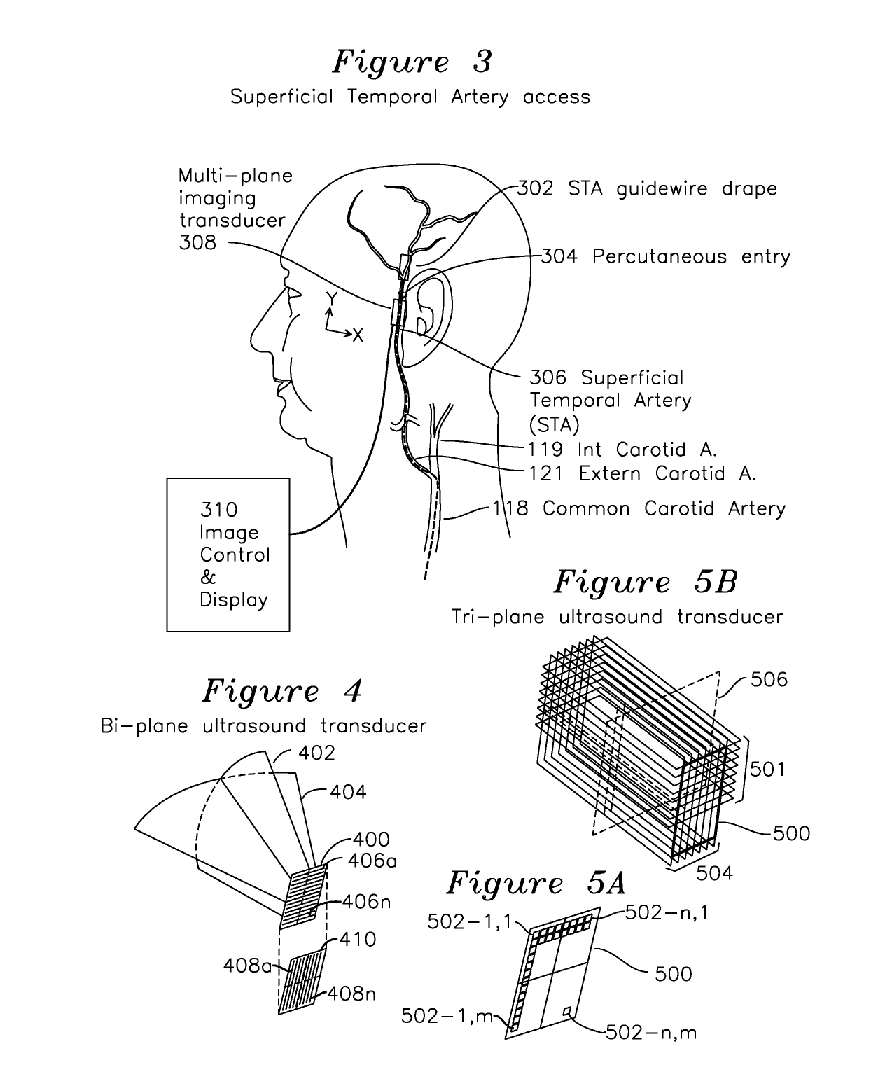

FIG. 3 in accordance with one embodiment of the invention.

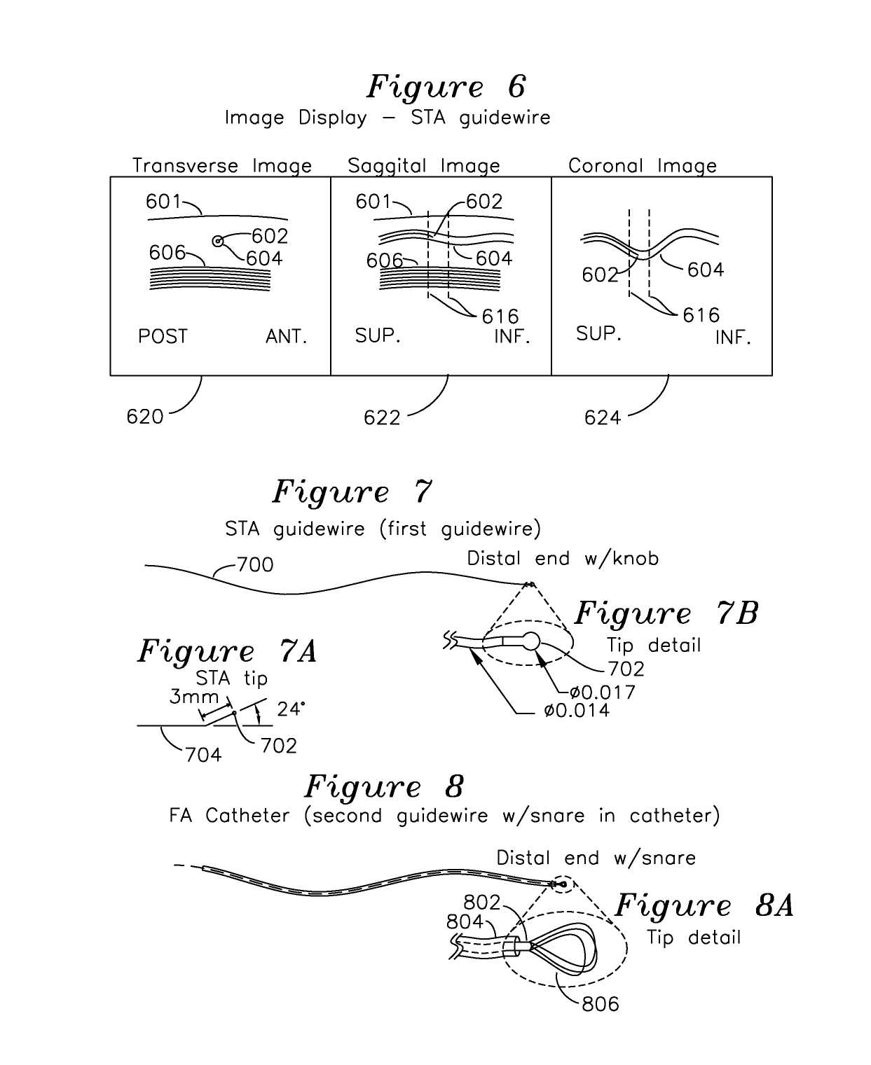

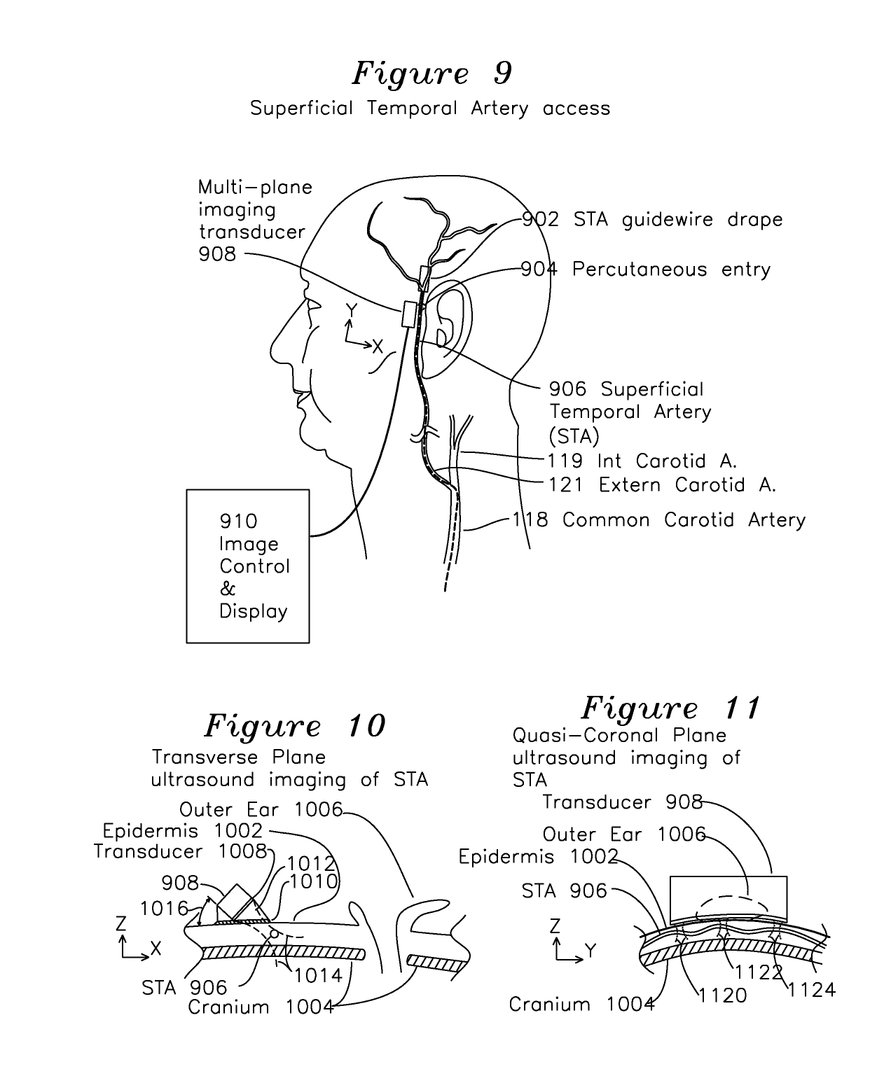

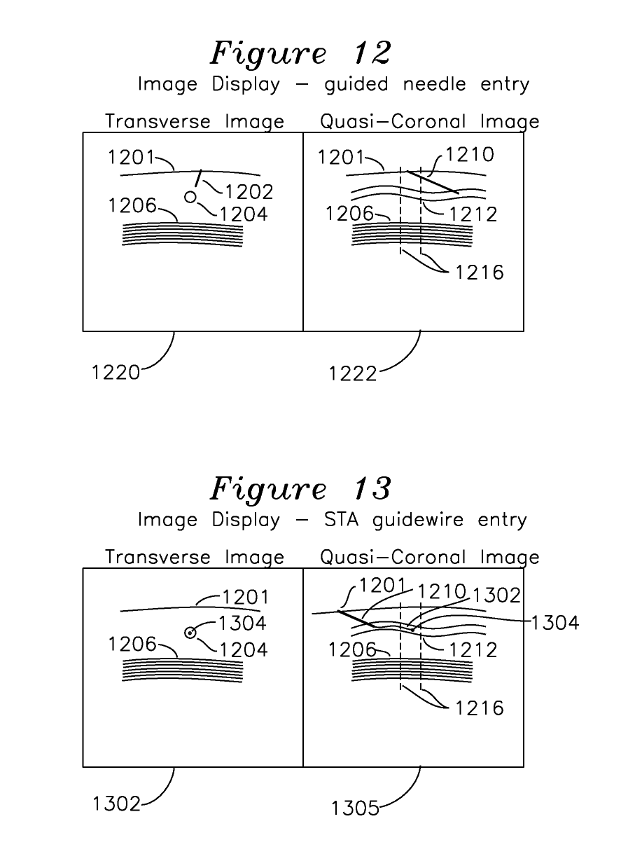

FIGS. 5A and 5B illustrate tri-plane acquisition of echo information from the transducer of FIG. 4 in accordance with one embodiment of the invention.

FIG. 6 illustrate an image display reconstructed from the acquisition data organized in FIG. 4 in accordance with one embodiment of the invention.

FIG. 7 illustrates an STA guidewire in accordance with one embodiment of the invention.

FIG. 7A is a detailed view of a guidewire tip of the guidewire of FIG. 7 in accordance with one embodiment of the invention.

FIG. 7B is a detailed view of a distal end knob of the guidewire of FIG. 7.

FIG. 8 illustrates an FA catheter including guidewire in accordance with one embodiment of the invention.

FIG. 8A is a detailed view of a distal tip of the FA catheter of FIG. 8.

FIG. 9 is a side-looking multi-plane imaging transducer applied adjacent to the STA of a subject in accordance with one embodiment of the invention.

FIG. 10 shows a transverse plane view of the transducer of FIG. 9 in accordance with one embodiment of the invention.

FIG. 11 shows a quasi-coronal image plane view of the transducer of FIG. 9 in accordance with one embodiment of the invention.

FIG. 12 illustrates a guided needle entry of the transducer of FIG. 9 in accordance with one embodiment of the invention.

FIG. 13 illustrates a guidewire in a vessel for the guided needle entry of FIG. 12 in accordance with one embodiment of the invention.

### DETAILED DESCRIPTION

The embodiments disclosed herein are only examples of the many possible advantageous uses and implementations of the innovative teachings presented herein. In general, statements made in the specification of the present application do not necessarily limit any of the various claimed embodiments. Moreover, some statements may apply to some inventive features but not to others. In general, unless otherwise indicated, singular elements may be in plural and vice versa with no loss of generality. In the drawings, like numerals refer to like parts through several views.

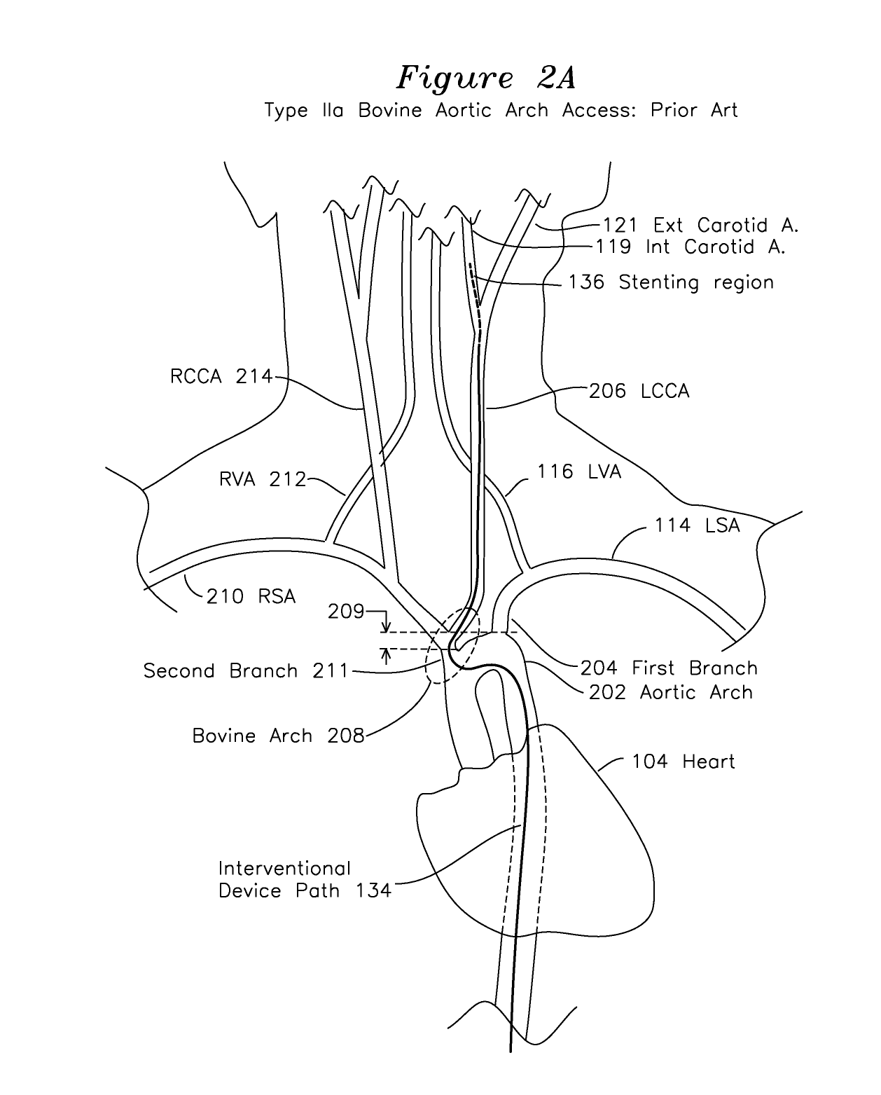

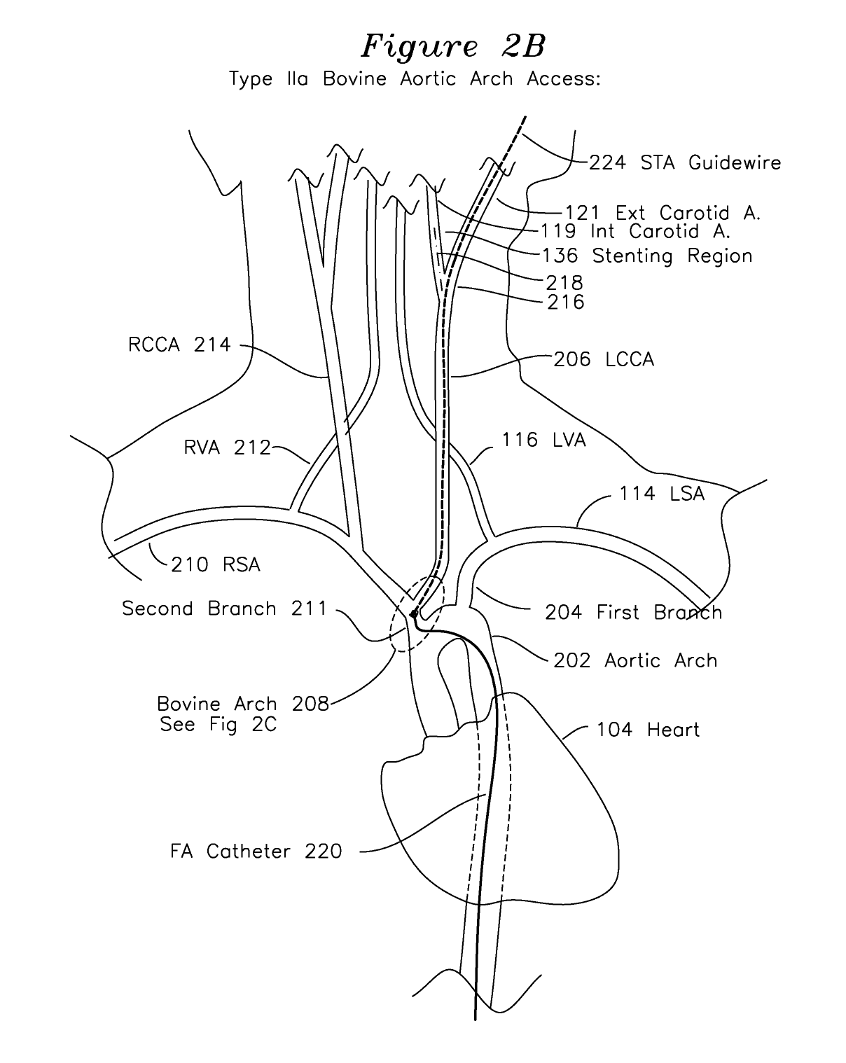

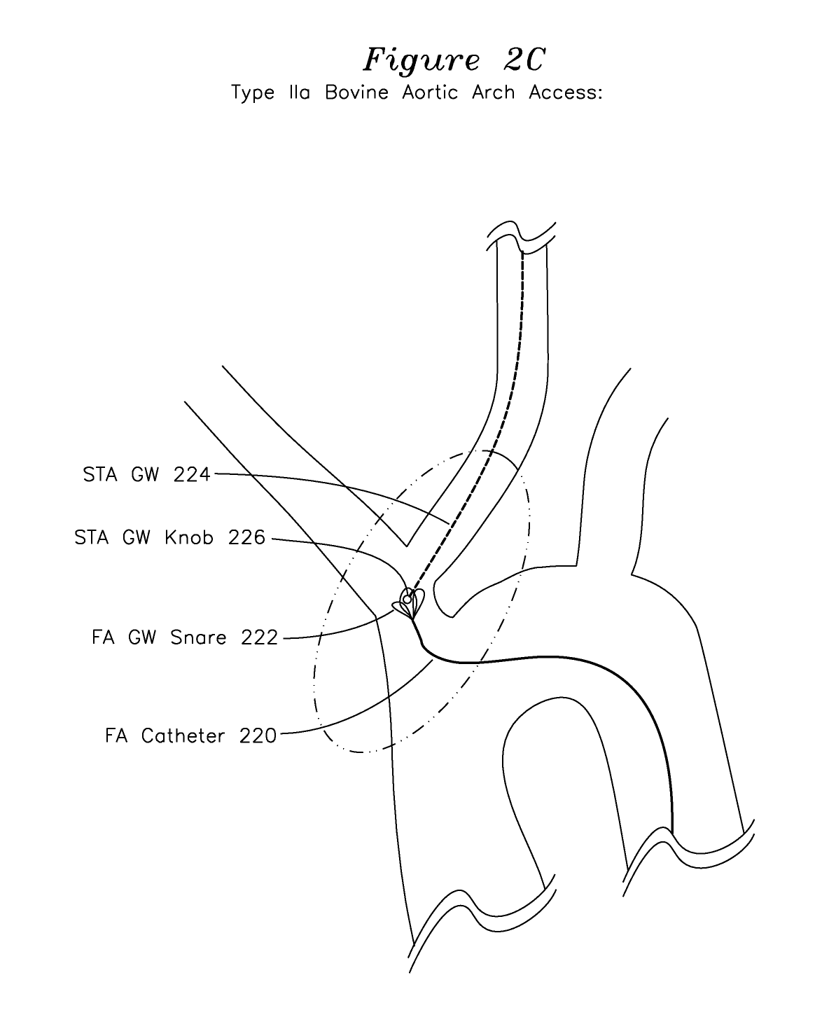

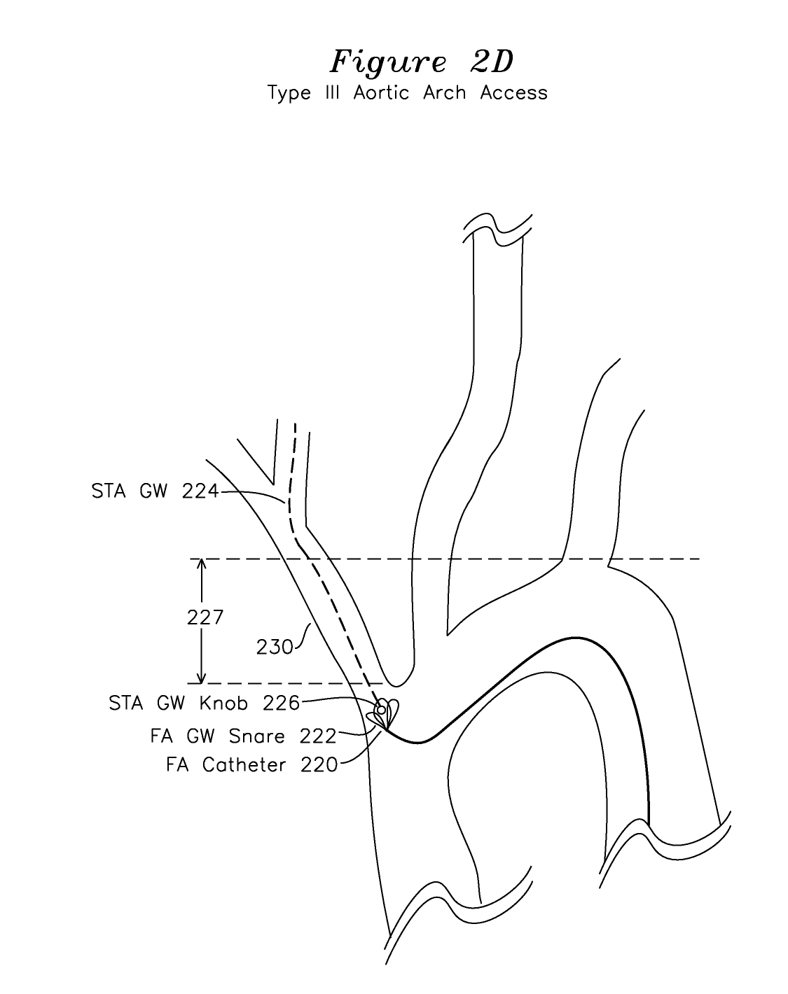

FIG. 2A illustrated the difficulty of gaining access to the internal carotid artery using the femoral artery and guiding through the sharp bends of a hostile aortic arch with a type II-A bovine variation.

FIG. 2B shows a first step in gaining stenting access to the left internal carotid artery (LCA) 119 by first inserting an STA guidewire 224 with a distal head or knob which has an increased diameter near the distal end compared to the guidewire diameter, and which provides an engagement volume for the FA catheter 220 to encircle and grab, as will be described later. The STA guidewire is introduced through a needle, such as a 21 gauge needle (not shown) having an inner diameter of 0.018 inch or more, and the STA guidewire diameter may be in the range of 0.014 to 0.016 inch with the distal knob having a diameter in the range of 0.017 to 0.018 inch, and with clearance to fit within the inner diameter of the needle. In one embodiment, the guidewire is 0.016 inch, and the knob is 0.018 inch. The knob of the STA guidewire 224 is threaded through the STA (306 of FIG. 3), which joins the external carotid artery 121, which joins the common carotid artery 206 leading to the second branch 211 of the type II-A bovine aortic arch variation 208. The knob end of STA guidewire 224 is guided until it is in a position where it may be engaged by the FA catheter 220, and guidance and snaring is done using conventional radiographic techniques known in interventional radiology.

FIG. 2C shows a magnified view of this engagement. As shown in FIG. 2C, FA catheter 220 snare end 222 encircles the knob 226 of STA guidewire 224, and the snare wires are withdrawn into FA catheter 220 to encircle and snare the STA guidewire 224 knob 226, after which the FA catheter 220 may be withdrawn from the femoral artery, thereby providing a single guidewire 224 with “through and through” access from the STA clear to the femoral artery. The STA guidewire 224 may subsequently be used as a scaffold to guide a sheath up to the bifurcation point 216 (of FIG. 2B) of the external and internal carotid artery, after which a stent guidewire may be guided along path 218 (of FIG. 2B) to the internal carotid artery 119 stent region.

FIG. 2D shows a hostile type III aortic arch as determined by retrograde distance 227, where the FA catheter 220 which would otherwise require difficult guidance to enter vessel 230 is guided to the vessel 230 entrance and snares STA guidewire 224 after passage through vessel 230, after which FA catheter 220 is withdrawn and pulling STA guidewire 224 along with it. Through and through access is provided using the remaining STA guidewire 224 to guide subsequent interventional devices into carotid artery accessible through vessel 230, thereby greatly simplifying the guidance of interventional devices into vessel 230.

FIG. 3 shows the STA guidewire dispenser 302, which may be a surgical drape containing the unused guidewire, and percutaneous entry 304 to the STA 306. A key problem in entering and guiding the guidewire into the STA is the fragility of the STA, as well as the STA size and tortuosity. Attempting to access the STA with a needle is difficult because of the tortuosity of the artery and small size of the STA guidewire. The guidewire needle may puncture clear through the STA, and the guidewire may puncture the artery or simply coil within the STA (and not advance), if not correctly guided. The first few centimeters of guiding are critical to the success of the STA guidewire entry, and the initial guidance requires the knowledge of the local axis position of the STA, in all three planes so that the needle entry may occur parallel to this local axis (at the point of needle entry during that initial part of the procedure) and that after entry of the guidewire, that the guidewire tip may be guided substantially parallel to the local vessel axis, where the local vessel axis is understood to be taken at the distal point of the guidewire, since the underlying problem is the vessel being navigated is tortuous and the local axis is specific to a particular point.

Visualizing the STA and relationship of the needle and guidewire in the STA is provided by multi-plane ultrasound probe 308 and image processor 310, where the imaging may be accomplished by using appropriate beam focusing to provide maximum resolution near the surface of the skin where the guidewire enters the STA, and the depth of focus may be dynamically changed by the image control 310 providing electronic focus to the array elements of transducer 308, thereby maintaining sharpest focus in the region of interest.

FIG. 4 shows the principles of operation of an ultrasound scanner. In a 2D phased array or linear array scanner, an imaging transducer 400 is formed from plurality of rectangular elements 406a through 406n, which are each processed through a temporal variable delay line on transmit and receive, thereby providing an image plane 402 which is formed from a sequence of individual line scans, each line formed by the intrinsic transducer elements 406a through 406n focus characteristics, and a selection of transmit and receive delays provided for beam steering for off-center angular sweep shown in the extents of 402, with the individual scan lines are placed together in a 2D memory array (not shown) to form an image plane such as 402. A second plurality of rectangular elements 408a through 408n is similarly energized in transducer 410 coincident with transducer 406 on transmit and receive to form a plurality of scan lines which analogously form scan plane 404. Transducers 408 and 410 are placed adjacent to each other so that each may form the scan planes 404 and 402, respectively. In this manner, it is possible to form a 2D biplane image which is formed from the image acquisition points of plane 402 and 404. Without rotating the transducer 400, the field of view is typically limited to the two image planes 402 and 404, and inclusion of regions outside these planes is accomplished by manually rotating the angle of transducer 400 about its long or short axis. The control of transducers 400 and 410 may be provided as a linear array (a subgroup of transducer elements are energized and signals received from a superset or subset of these elements to form the image), or as a phased array (with each element receiving an appropriate phase delay to provide beam steering in the scan plane to form the image), as is known in the art of ultrasound array imaging.

FIG. 5A shows a variant 500 of the transducer 400 shown in FIG. 4, and provides for forming the corresponding elements 406a through 406n and 408a through 408n using a two dimensional array of individual elements along one axis, shown as 502-1,1 to 502-n,1 and elements arranged on the perpendicular axis shown as 501-1,1 through 502-1,m, thereby forming an m×n array of elements, each of which is able to transmit and receive using a programmed delay. In this manner, replacing the linear arrays 404 and 410 of FIG. 4 with array 500 of FIG. 5, individual delays may be generated to cause image plane 404 to be steered up and down (not shown) as well as from side to side as shown, and image plane 402 may be steered from side to side (not shown) as well as up and down as shown, thereby forming a 3D array of acquired data, using either all vertical image data planes 504, or all horizontal image data planes 501. These acquisition image planes are shown as parallel planes in FIG. 5B for simplicity in understanding the invention, although it is known in the art of scan conversion that these may be polar arrays of image data which are converted to rectangular arrays using methods known in the art of ultrasound imaging and scan conversion. As is known in the art of ultrasound imaging, for a phased array scanner, the image planes would typically be azimuthally separated from each other by an included angle about a common axis, and for a linear array scanner, the image planes would be formed by groups of elements, forming substantially parallel planes as shown in FIG. 5B. For simplicity, the image planes indicating separately acquired images are shown in FIG. 5 as parallel planes. In one embodiment of the invention, primary image data is acquired for each of the sets of planes 501 and 504, and a third image plane 506 is synthetically created by using data from either the image plane set 502 or the image plane set 504, such as by selecting data corresponding to a separation distance from the transducer 500 face. It is also known that, by quadrature baseband mixing the return echoes from the transducer after beam focusing, flow velocity may be determined throughout the volume and displayed with color intensity associated with flow rate displayed along with the ultrasound image in what is known as color flow Doppler. It is also possible to establish a sampling window by selecting a particular image depth (proportional to delay from transmit time) and thereby provide pulsed Doppler flow as an audible or time-domain plot or computed value, which is known as pulsed Doppler or gate-delay Doppler. In color flow Doppler, this provides for clear identification of blood vessels containing blood flow from static structures, and for pulsed Doppler provides quantitative information about flow velocity.

FIG. 6 shows three views constructed from image plane data of FIG. 5B. For a multi-plane transducer 308 positioned directly over the STA as shown in FIG. 3, and with a transducer long axis aligned with the local axis of the STA below the transducer 308, and using the m×n array of FIG. 5A, the transverse image 620 may be may be formed using a selected one of image data planes 501, and the saggital image 622 may be formed using a selected one of the image data planes 504. Coronal image 624 may be constructed using image data from the vertical planes 504 or horizontal image planes 501 which include the desired range of depth such as points corresponding to plane 506 to generate coronal image display 624. Accordingly, the transverse display 620 show skin surface 601, STA 604, STA guidewire 602, and cranium 606, and may be labeled with posterior and anterior views for orientation. Saggital image 622 shows skin surface 601, STA 604, guidewire 602, and cranium 606. Coronal image 624 shows the tortuous meandering of the STA 604 and guidewire 602. The combination of images 620, 622, and 624 thereby provides the information needed by an interventional radiologist to guide the STA guidewire 602 through the tortuous regions of the STA required for navigation to the common carotid artery as described previously. In one embodiment of the invention, color flow imaging or pulsed Doppler flow information is provided within the images 620, 622, or 624 to further highlight the extent of vessel 604 or establish flow rates in the vessel, which may be used for guidance of the needle into the vessel, or for guidance of the guidewire through the vessel. In this view, the interior regions of STA 604 would indicate instantaneous flow, which would be useful for identifying the STA and its viability for use based on blood flow velocity from the color Doppler image in the vessel or pulsed Doppler information measured at the depth of the vessel from the displayed image.

FIG. 7 shows additional details of the STA guidewire 700. FIG. 7A shows the formed guidewire shape of the distal tip of the STA, which includes a 3 mm extent at the tip which is bent at an angle as shown, but may be within the range from 15° to 45°. STA guidewire 704 terminates in knob 702, shown in magnified detail FIG. 7B. The STA guidewire 704 has a diameter of about 0.014 inch, although may be within the range 0.014 inch to 0.016 inch, and knob 702 has a diameter of within the range of about 0.017 to 0.018 inch, but preferably has a diameter of about 0.017 inch to fit within the inner diameter of a 21 gauge needle for entry into the STA through the needle. In one embodiment of the invention, multiple guidewire bends are present, including for example a first bend at the distal tip with a bend angle from about 10 to 45 degrees and a second bend in the range of about 10 to 45 degrees separated from the first bend by about 1-10 mm and placed on the opposite side from the knob end. The first and second bends may be about 20 degrees and the two bends may be separated from each other by less than 10 mm.

FIG. 8 shows an example FA catheter with a snare inserted, which as previously described and shown in magnified detail FIG. 8A, comprises outer catheter 804 and inner guidewire 802 with snare 806. The FA catheter structures are in the range of 4 French to 8 French sheath enclosing an FA guidewire in the range of about 0.018 to 0.038 inch in a lumen formed into the sheath which exceeds the guidewire diameter. The snare may be formed from one or more wires which form one or more loops 806 with a diameter range of about 15-45 mm which may be used for snaring the STA guidewire in the aortic arch, or having a loop diameter in the range of about 4 mm-8 mm for snaring the STA guidewire in the external carotid artery.

In another embodiment of the invention shown in FIG. 9, a multi-plane ultrasound imaging probe 908 is coupled to an ultrasound imaging controller and display 910. The ultrasound probe 908 has special characteristics and performance related to imaging the STA 906 and allowing the insertion of a needle into the STA during imaging. The embodiment of FIG. 9 provides for a simpler biplane imaging system than the 3D imaging system previously described for FIG. 3, and also provides for an angular offset which provides two planes of image viewing, a transverse plane as before, and a quasi-coronal plane view which captures the tortuosity of the meandering of the STA in the coronal plane using a primary imaging plane, rather than constructing the coronal image from synthesized image data from primary 2D imaging planes as was described for FIG. 5B.

FIG. 10 shows a transverse plane view of a subject with relationship to imaging probe 908. For the purposes of the current invention, and in accordance with how a clinician typically would use the device, the “transverse view” is a view which provides a cross section of the STA through its local axis. Accordingly, maintaining the “transverse view” as the clinician follows the progress of the guidewire in the STA may require rotating the probe 908 in a plane parallel to the epidermis 1002 (in the X-Y plane, local to the skin surface 1002) The ultrasound probe 908 includes transducer 1008 which has the characteristic of imaging in two substantially orthogonal planes as described in FIG. 4, and includes an angular offset to provide for imaging to the side of the transducer to allow simultaneous needle access at the percutaneous entry 904 and imaging access with probe 908. In one embodiment of the invention, transducer face 1008 combines two perpendicular arrays of elements which operate at different intervals of time to provide the required two planes of imaging, and the probe includes an interface which is angled to the skin surface 1002. The angle between a line normal to the face of the transducer 1008 to the local X-Y plane may be in the range of 30 degrees to 90 degrees. A basic physical tradeoff is between imaging access to the STA and needle access to the STA. As shown in FIG. 10, the region above the STA is exposed during imaging with an example angle of 45 degrees, although other angles between the transducer and skin surface 1002 are possible. A coupling gel 1010 may be provided for minimizing acoustic discontinuities between probe transducer 1008 and skin surface 1002, and angled offset coupler 1012 may be fabricated from a flexible material which provides minimal internal reflection or includes a gradient in refractive index to provides acoustic beam focusing to provide minimal beam width at the depth of the STA 906. The transducer 908 configuration of FIG. 10 thereby provides simultaneous access to the STA 906 in the region above for entry and imaging of the STA guidewire, as well as an image transverse to the local axis of the STA, shown in the X-Z plane of FIG. 10. Ear 1006 and cranium 1004 are shown for reference. Lines 1014 indicate the trend of general focus characteristics of the ultrasound beam waist in the range of the vessel, although the actual beam characteristic which provides fundamental spatial resolution is governed by the diameter of the STA being imaged, and may be adjusted by the image control 910.

FIG. 11 shows a quasi-coronal plane view, where the quasi-coronal plane is orthogonal to the transverse plane of FIG. 4 and with an angle of 30 to 90 degrees, preferably 45 degrees, to the skin 1002, and shown as the Y-Z plane. The transducer 908 has a rectangular aspect ratio, which provides linear array imaging by actuating groups of individual transducers forming 908 as described in FIG. 4, thereby allowing a quasi-coronal quasi-saggital image of STA 306. An decreased angle 1016 less than 45 degrees is preferred for viewing the tortuosity (meandering) of the STA and associated guiding of the STA guidewire, since the STA meanders coronally in the tissues adjacent to the cranium, and a small angle 1016 provides for a best direct coronal view of this tortuosity. Beam profiles 1130, 1122, and 1124 indicate the progression of elements such as for a linear array, where groups of elements are energized in succession to form the rectangular array of data used to form the beam. Transducer face 908 is shown as a curved array to match the curvature of the cranium 1004, however the transducer face may be planar, or any shape which provides suitable acoustic coupling to the STA for imaging.

FIG. 12 shows an example image display for the STA guidewire intervention of FIGS. 9, 10, and 11. The image pair 1220, 1222 of FIG. 12 indicates a simultaneous transverse image 1220 and quasi-coronal image 1222, representing the views afforded by the transducer geometry shown in FIGS. 10 and 11. The example transverse image 1220 displays ultrasound echoes indicating skin layer 1201, highly reflective cranium 1206, STA vessel 1204, and needle 1202. View 1220 would appear to show that needle 1202 has not contacted STA vessel 1204, and this example illustrates the importance of the present multi-plane scan invention, as in quasi-coronal view 1222 which is simultaneously presented, it is clear that the needle 1210 has already punctured the STA 1212 vessel first wall, but is simply out of the range of transverse focus shown by cursor lines 1216.

FIG. 13 indicates an example image display where the STA guidewire 1304 is introduced following proper location of the guidewire needle in FIG. 12 into STA 1204. The image probe is moved to along the path of the STA, so the transverse needle image is no longer visible in the image 1302 field of view, but may be seen 1210 in the quasi-coronal image 1304. Quasi-coronal image 1304 shows a view of the introduced STA guidewire 1302 and STA guidewire knob 1304, which also appears in the transverse image 1302, as it is within the transverse focus extents 1216 shown in quasi-coronal image 1305.

In another embodiment of the invention, the knob 702 of FIG. 7B may be treated with an axial winding of wire and generate an electromagnetic field, which electromagnetic field may be used in combination with the imaging techniques described herein to provide guidance of the STA guidewire within the STA using positional information provided by the electromagnetic field information.

In one embodiment of the invention, a multiplane reference view is provided with the multiple planes intersecting at a reference point which is aligned to guidewire tip 1304 of FIG. 13 (or needle 1202 tip of FIG. 12) such that image 1302 is selected to include this reference point, thereby showing the knob tip of the STA guidewire (or tip of the needle navigating to the tortuous artery) with visual extents 1216 provided to ensure the views are adjusted to follow the guidewire knob 1304 or needle 1202 tip. In one embodiment of the invention, this tracking is provided by an electromagnetic winding provided on the axis of the needle and guidewire in combination a proximal magnetic field detector which resolves rotation and translation of the needle or guidewire.

Because of the small size of the STA, a high frequency ultrasound transducer is preferred. It is known that the axial response of an individual scan line is associated with the temporal response of a single imaging element, and that acoustic energy propagates through the body at a rate of approximately 1.5 mm per microsecond. A typical ultrasound reflection represents 3-5 cycles at the center frequency of the transducer, and accordingly, a 10 Mhz piezoelectric transducer crystal has a temporal response of 500 us, corresponding to 0.5 mm of resolution, which is on the lower end of required resolution of the 3-4 mm STA. Accordingly, a transducer with a frequency greater than 10 Mhz is preferred, with the imaging depth limited by the Rayleigh scattering attenuation on the order of Idb\cm\Mhz, corresponding to a 60 db SNR (relative to transmit power) imaging depth of 60 mm at 10 Mhz, or 20 mm at 30 Mhz. Accordingly, ultrasound transducer frequency ranges from 10 to 30 Mhz are expected to be preferable to provide adequate resolution as the low frequency limit and adequate penetration at the high frequency limit. In one embodiment of the invention the lower operation frequency begins at 7 MHz.

In one embodiment of the invention related to the process for placement of a stent, the STA guidewire is guided through the STA using the bi-plane ultrasound imager, the FA catheter with a bent-tip guidewire installed is introduced into the femoral artery and guided to the aortic arch region where it can snare the knob end of the STA guidewire. The locating and snaring of the knob end of the STA guidewire is done using fluoroscopic imaging, as is known in the art. The guidewire is withdrawn from the FA catheter and replaced by a snare, which is advanced through the FA catheter into the aortic arch region where it snares the knob end of the STA catheter. The STA guidewire is pulled using the snare into the FA catheter which is then pulled out through the common femoral artery access, thereby providing “through and through” access. A long guiding sleeve or sheath is then advanced over the STA guidewire into the distal common carotid artery. A second wire is now used and subsequently guided through the internal carotid artery stenosis (narrowing) until it reaches a desired region for placement of the embolic protection device. The stent is advanced over this second guidewire. The stent is expanded over the wire below the embolic protection device at the site of carotid stenosis. The embolic protection device is removed followed by removal of the second guidewire. The knob guidewire may be removed at the very end of the procedure.

In another embodiment of the invention, the STA guidewire is snared in the ECA or the CCA by initially guiding the FA catheter to the ECA or CCA, respectively.

In another embodiment of the invention, the apparatus and method may be applied to the lower extremities. For example, in a subject with a blockage in the legs such as in the tibial or pedal artery (such as diabetics) or subjects with advanced infrapopliteal occlusions, it is possible to use the multi-plane imager to guide a fine steerable guidewire through the tortuous vessels in the feet and slightly distal to the occlusion site, then thread a 3 French or 4 French catheter over the fine steerable wire, withdraw the fine steerable wire from the sheath, and then introduce a stiff guidewire in the range of about 0.014 inch to 0.018 inch through the blockage, the stiff guidewire having a knob end with a knob diameter greater than the stiff guidewire diameter, and thereafter snaring the knob using an FA catheter introduced from the femoral artery and guided distally to the stiff guidewire. The stiff guidewire is then snared or guided directly into the FA catheter (if possible), and the guidewire is withdrawn or advanced through the FA catheter, thereby providing through and through access as a platform for subsequent procedures.

In another embodiment of the invention, the STA guidewire is introduced as before, however the FA catheter procedure is slightly different. In this embodiment, a sheath and first guidewire are introduced together into the femoral artery to the aortic arch region, the guidewire guiding the sheath to the desired location of the aortic arch, after which the FA catheter sleeve alone is threaded over the guidewire to the snaring location, after which the guidewire is removed and replaced with the snare such as FIG. 8A which is used to capture the knob of the STA guidewire as described earlier.

One of ordinary skill in the art would readily appreciate that embodiments of the apparatus may be used for endovascular stroke intervention and other neuro-interventional procedures in hostile aortic arches. Also this device can be helpful for quick and reliable radial artery access for any type of Endovascular interventional and pedal artery access for limb salvage procedures.

All examples and conditional language recited herein are intended for pedagogical purposes to aid the reader in understanding the principles of the invention and the concepts contributed by the inventor to furthering the art, and are to be construed as being without limitation to such specifically recited examples and conditions. Moreover, all statements herein reciting principles, aspects, and embodiments of the invention, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

What is claimed is:

1. An apparatus for the placement of a stent, the apparatus comprising:
a multi-plane imaging transducer that generates a plurality of planes of image data from ultrasound echo information, each of the plurality of planes formed from at least one scan line, each scan line generated by at least one transducer element selected from a plurality of transducer elements forming the multi-plane imaging transducer, each transducer element activated through a temporal delay, the plurality of planes including at least a first plane and a second plane wherein the first plane is orthogonal to the second plane.

2. The apparatus of claim 1, further comprising a Superficial Temporal Artery (STA) guidewire associated with the multi-plane imaging transducer, the STA guidewire having a distal end, the distal end formed into a knob and having a bend that provides for steering through rotation of the STA guidewire.

3. The apparatus of claim 2, wherein the STA guidewire has a diameter in the range of 0.014 inch to 0.016 inch.

4. The apparatus of claim 2, wherein the STA guidewire knob has a diameter in the range of 0.017 inch to 0.018 inch.

5. The apparatus of claim 2, wherein the STA guidewire has a diameter of 0.016 inch and the distal knob has a diameter of 0.018 inch.

6. The apparatus of claim 2, wherein the distal end of the STA guidewire includes a bend angle in the range of 10 degrees to 45 degrees within a distance range of 3 mm from a distal end of the knob.

7. The apparatus of claim 2, wherein the multi-plane imaging transducer is further associated with a femoral artery (FA) catheter kit, the catheter kit comprising a catheter and a guidewire with a distal end having a snare for mechanical engagement with the knob.

8. The apparatus of claim 7, wherein the guidewire of the FA catheter kit has a diameter in the range of 0.018 to 0.038 inches.

9. The apparatus of claim 7, wherein the catheter of the FA catheter kit has a diameter in the range of 4 French to 8 French.

10. The apparatus of claim 1, further comprising:
an image control that generates a plane of coronal image data from the plurality of planes by selecting image data in each plane of the plurality of planes which correspond to a separation distance from the multi-plane imaging transducer.

11. The apparatus of claim 10, wherein the image control further comprises:
a display processor coupled to the multi-plane imaging transducer and to the image control for simultaneous displays of a transverse view, a saggital view, and a coronal view derived from the planes of image data.

12. The apparatus of claim 1, wherein the multi-plane imaging transducer further comprises:
a rectangular array of transducer elements having ‘m’ transducer elements in a first axis of the rectangular array and having ‘n’ transducer elements in a second axis of the rectangular array, the second axis being perpendicular to the first axis.

13. The apparatus of claim 1, wherein the multi-plane imaging transducer further comprises:
a first array of ‘m’ transducer elements arranged along a first axis of the multi-plane imaging transducer which is adjacent to a second array of ‘n’ transducer elements arranged along a second axis the multi-plane imaging, the second axis being perpendicular to the first axis.

14. The apparatus of claim 1, wherein the multi-plane imaging transducer further comprises:
a piezoelectric array of elements adapted to operate in a frequency range of 10 Mhz to 30 Mhz.

15. The apparatus of claim 1, wherein the multi-plane imaging transducer further comprises:
a piezoelectric array of elements adapted to operate in a frequency range of 7 Mhz to 30 Mhz.

16. The apparatus of claim 1, wherein the first plane comprises a transverse plane and the second plane comprises a saggital plane.

17. A method for positioning a stent in a carotid artery of a subject, the method comprising:
inserting a femoral artery (FA) catheter into a femoral artery of the subject, the FA catheter further allowing an insertion of a snare after positioning;
advancing the FA catheter to within a snare extent within an aortic arch of the subject;
capturing ultrasound transverse and coronal images captured by a multi-plane ultrasound transducer;
guiding a Superficial Temporal Artery (STA) guidewire through a tortuous region of a superficial temporal artery responsive of the ultrasound transverse and coronal images, the STA guidewire further comprising an integral knob engagement with the snare placed in the FA catheter;
manipulating the STA guidewire and the FA catheter until the STA guidewire knob is engaged with the snare introduced into the FA catheter;
removing the FA guidewire;
positioning a long guiding sleeve or sheath at a desired location using the STA guidewire;
advancing a stent to an installation site of the carotid artery using the long guiding sleeve or sheath; and
installing the stent by expanding and securing the stent to the carotid artery.

18. The method of claim 17, wherein capturing ultrasound transverse and coronal images further comprises:
selecting image data in each plane of the plurality of planes which correspond to a separation distance from the multi-plane imaging transducer.

19. The method of claim 18, wherein capturing ultrasound transverse and coronal images further comprises:
displaying simultaneously of a transverse view, a saggital view, and a coronal view derived from the planes of image data.

20. The method of claim 17, wherein capturing ultrasound transverse and coronal images further comprises:
operating the multi-plane ultrasound transducer in a frequency range of 10 Mhz to 30 Mhz.

21. The method of claim 17, wherein capturing ultrasound transverse and coronal images further comprises:
operating the multi-plane ultrasound transducer in a frequency range of 7 Mhz to 30 Mhz.

22. A guidewire installation method for introducing a single guidewire through a first tortuous vessel and providing the guidewire to a second vessel having a larger diameter than the tortuous vessel, the process comprising:
capturing by a multi-plane ultrasound transducer image information for determination of at least a local axis of the tortuous vessel;
guiding a needle having an inner diameter and an outer diameter to enter the tortuous vessel at an angle substantially parallel to the local axis of the tortuous vessel at a point of entry to the tortuous vessel;
threading through the needle and into the vessel a first guidewire, the first guidewire further comprising a distal knob for snaring and a bend for steering the guidewire through the tortuous vessel;
wherein at least one of the needle inserting step or the first guidewire threading utilizing image information for guiding the needle or the first guidewire along the local axis;
introducing a catheter, the catheter having an inner diameter and an outer diameter and a second guidewire inserted into the inner diameter, into the second vessel and towards the first guidewire to within a snaring region;
replacing the second guidewire with a snare wire, the snare wire encircling the first guidewire knob;
removing the snare wire and the first guidewire from the second vessel, thereby providing the first guidewire as a through and through guidewire to support a subsequent procedure.

23. The guidewire installation method of claim 22, wherein the image information comprises at least a simultaneous coronal view and transverse view, the coronal view and transverse view indicating a common plane for indicating a guidance direction for the guidewire.

24. The guidewire installation method of claim 22, wherein the needle is a 21 gauge needle.

25. The guidewire installation method of claim 22, wherein the first tortuous vessel is a Superficial Temporal Artery (STA).

26. The guidewire installation method of claim 22, wherein the second vessel is a femoral artery (FA).

27. The guidewire installation method of claim 22, wherein the first tortuous vessel is a surface vessel of a foot of the subject.

28. The guidewire installation method of claim 22, wherein the second vessel is at least one of a femoral artery, a tibial artery, or a pedal artery, of the subject.

29. The guidewire installation method of claim 22, wherein the first guidewire has a diameter of 0.014 to 0.016 inch.

30. The guidewire installation method of claim 29, wherein the knob has a diameter larger than the first guidewire diameter and further in the range 0.017 to 0.018 inch.

31. The guidewire installation method of claim 22, wherein the catheter has a diameter in the range of 4 French to 7 French.

32. The guidewire installation method of claim 22, wherein the subsequent procedure is at least one of: a stent installation or a balloon angioplasty.

33. An imaging apparatus for navigating an intravascular device, the apparatus comprising:
a Superficial Temporal Artery (STA) guidewire with a distal end, the distal end formed into a knob and having a bend providing for steering through rotation of the STA guidewire;
a femoral artery (FA) catheter kit comprising a catheter and a guidewire with a distal end having a snare for mechanical engagement with the knob;
a multi-plane imaging ultrasound transducer that generates a plurality of planes of image data from ultrasound echo information, each plane formed from at least one scan line, each scan line generated by at least one transducer element selected from a plurality of transducer elements forming the multi-plane imaging ultrasound transducer, each transducer element activated through a temporal delay, the plurality of planes including at least a first plane and a second plane, the second plane being orthogonal to the first plane.

34. The apparatus of claim 33, wherein the first plane comprises a transverse plane and the second plane comprises a saggital plane.

35. The apparatus of claim 33, wherein the multi-plane imaging transducer further comprises:
a rectangular array of transducer elements having ‘m’ transducer elements in a first axis of the rectangular array and having ‘n’ transducer elements in a second axis of the rectangular array, the second axis being perpendicular to the first axis.

36. The apparatus of claim 33, wherein the multi-plane imaging transducer further comprises:
a first array of ‘m’ transducer elements arranged along a first axis of the multi-plane imaging transducer which is adjacent to a second array of ‘n’ transducer elements arranged along a second axis the multi-plane imaging, the second axis being perpendicular to the first axis.

37. The apparatus of claim 33, wherein the multi-plane imaging transducer further comprises:
a piezoelectric array of elements adapted to operate in a frequency range of 10 Mhz to 30 Mhz.

38. The apparatus of claim 33, wherein the multi-plane imaging transducer further comprises:
a piezoelectric array of elements adapted to operate in a frequency range of 7 Mhz to 30 Mhz.

39. An imaging apparatus for navigating an intravascular device, the apparatus comprising:
a Superficial Temporal Artery (STA) guidewire with a distal end, the distal end formed into a knob and having a bend providing for steering through rotation of the STA guidewire;
a femoral artery (FA) catheter kit comprising a catheter and a guidewire with a distal end having a snare for mechanical engagement with the knob;
a biplane ultrasound imaging transducer that generates two planes of image data from ultrasound echo information, each plane formed from at least one scan line, each scan line generated by at least one group of individual transducers selected from a plurality of groups of individual transducers forming the biplane ultrasound imaging transducer, each group of individual transducers energized in succession, the two planes orthogonal to one another, the biplane biplane ultrasound imaging transducer angled at 30 degrees to 90 degrees to a subject's skin.

40. The apparatus of claim 39, wherein the biplane imaging transducer further comprising:
a piezoelectric array of elements adapted to operate in a frequency range of 10 Mhz to 30 Mhz.

41. The apparatus of claim 39, wherein the biplane imaging transducer further comprising:
a piezoelectric array of elements adapted to operate in a frequency range of 7 Mhz to 30 Mhz.

42. An imaging kit for navigating an intravascular device, the kit comprising:
a Superficial Temporal Artery (STA) guidewire with a distal end, the distal end formed into a knob and having a bend providing for steering through rotation of the STA guidewire;
a femoral artery (FA) catheter and guidewire with a distal end having a snare for mechanical engagement with the knob;
a multi-plane imaging transducer configured to generate a plurality of planes of image data from ultrasound echo information, each plane formed from at least one scan line, each scan line generated by at least one transducer element selected from a plurality of transducer elements forming the multi-plane imaging transducer, each transducer element activated through a temporal delay, the plurality of planes including at least a first plane and a second plane, the second plane being orthogonal to the first plane.

43. The apparatus of claim 42, wherein the first plane comprises a transverse plane and the second plane comprises a saggital plane.

44. The apparatus of claim 42, wherein the multi-plane imaging transducer further comprises:
a rectangular array of transducer elements having ‘m’ transducer elements in a first axis of the rectangular array and having ‘n’ transducer elements in a second axis of the rectangular array, the second axis being perpendicular to the first axis.

45. The apparatus of claim 42, wherein the multi-plane imaging transducer further comprises:
a first array of ‘m’ transducer elements arranged along a first axis of the multi-plane imaging transducer which is adjacent to a second array of ‘n’ transducer elements arranged along a second axis the multi-plane imaging, the second axis being perpendicular to the first axis.

46. The apparatus of claim 42, wherein the multi-plane imaging transducer further comprises:
a piezoelectric array of elements adapted to operate in a frequency range of 10 Mhz to 30 Mhz.

47. The apparatus of claim 42, wherein the multi-plane imaging transducer further comprises:
a piezoelectric array of elements adapted to operate in a frequency range of 7 Mhz to 30 Mhz.
